(19) United States
(12) Patent Application Publication
(10) Pub. No.: US 2022/0151708 A1
(43) Pub. Date: May 19, 2022

(54) Endoscopic Guidance Using Neural Networks

(71) Applicant: The Board of Regents of the University of Oklahoma, Norman, OK (US)

(72) Inventors: Qinggong Tang, Norman, OK (US); Chongle Pan, Norman, OK (US); Chen Wang, Norman, OK (US)

(21) Appl. No.: 17/530,131

(22) Filed: Nov. 18, 2021

Related U.S. Application Data
(60) Provisional application No. 63/115,452, filed on Nov. 18, 2020.

(51) Int. Cl. A61B 34/20; G06T 7/00

(52) U.S. Cl. CPC A61B 34/20; G06T 7/0012; G06T 2207/10068; G06T 2207/20081; A61B 2034/2055; G06T 2207/20084; G06T 2207/30084; G06T 2207/30101; G06T 2207/10101

(57) ABSTRACT

A method comprises obtaining an endoscope; obtaining a needle; inserting the endoscope into the needle to obtain a system; inserting the system into an animal body; and distinguishing components of the animal body using the endoscope and while the system remains in the animal body. A system comprises a needle; and an endoscope inserted into the needle and configured to: store a convolutional neural network (CNN); distinguish among a cortex of a kidney of an animal body, a medulla of the kidney, and a calyx of the kidney using the CNN; and distinguish between vascular tissue and non-vascular tissue in the animal body using the CNN.

### CROSS-REFERENCE TO RELATED APPLICATIONS

This claims priority to U.S. Prov. Patent App. No. 63/115,452 filed on Nov. 18, 2020, which is incorporated by reference.

### BACKGROUND

PCN was first described in 1955 as a minimally-invasive, x-ray guided procedure in patients with hydronephrosis. PCN needle placement has since become a valuable medical resource for minimally-invasive access to the renal collecting system for drainage, urine diversion, the first step for PCNL surgery, and other therapeutic intervention, especially when the transurethral access of surgical tools into the urological system is difficult or impossible. Despite being a common urological procedure, it remains technically challenging to insert the PCN needle correctly in the right place. During PCN, a needle penetrates the cortex and medulla of the kidney to reach the renal pelvis. Conventional imaging modalities have been used in PCN puncture. Ultrasound technique, as a commonly used medical diagnostic imaging method, has been utilized in PCN surgery for decades. Additionally, fluoroscopy and CT are also employed in PCN guidance, and sometimes they are used with ultrasonography simultaneously. However, due to the limited spatial resolution, these standard imaging modalities have proven to be inadequate for accurately locating the needle tip position. The failure rate of PCN needle placement is up to 18%, especially in non-dilated systems or for complex stone diseases. Failure of inserting the needle into the targeted location in the kidney through a suitable route might result in severe complications. Moreover, fluoroscopy has no soft tissue contrast and, therefore, cannot differentiate critical tissues, such as blood vessels, which are important to avoid during the needle insertion. Rupture of renal blood vessels by needle penetrations can cause bleeding. Temporary bleeding after PCN placement occurs in ˜95% of cases. Retroperitoneal hematomas have been found in 13% of cases. When PCNL is followed, hemorrhage requiring transfusion increases to 12-14% of the patients. Additionally, needle punctures during PCN can lead to infectious complications such as fever or sepsis, thoracic complications like pneumothorax or hydrothorax, and other complications like urine leak or rupture of the pelvicalyceal system.

Therefore, the selection of position and route of the puncture is important in PCN needle placement. It is recommended to insert the needle into the renal calyx through calyx papilla because fewer blood vessels are distributed on this route, leading to a lower possibility of vascular injury. Nevertheless, it is always difficult, even for experienced urologists, to precisely identify this preferred inserting route in complicated clinical settings. If PCN puncture is executed multiple times, the likelihood of renal injury increases and the operational time lengthens, resulting in higher risks of complications.

To better guide PCN needle placement, substantive research work has been done to improve the current guidance practice. Ultrasound with technical improvements in many aspects has been utilized. For instance, contrast-enhanced ultrasound has been proved to be a potential modality in the guidance of PCN puncture. Tracked ultrasonography snapshots are also a promising method to improve the needle guidance. In order to resolve bleeding during needle puncture, combined B-mode and color Doppler ultrasonography has been applied in PCN surgeries and it provides promising efficiency in decreasing major hemorrhage incidence. Moreover, developments in other techniques such as cone-beam CT, retrograde ureteroscopy, and magnetic field-based navigation devices have been utilized to improve the guidance of PCN needle access. On the other hand, an endoscope can be assembled within a PCN needle and effectively improve the precision of PCN needle punctures, resulting in lower risks of complications and fewer times of insertions. However, most of the conventional endoscopic techniques involving CCD cameras can only provide 2D information and cannot detect subsurface tissue before the needle tip damages it. Thus, there is a critical need to develop new guiding techniques which have depth-resolved capability for PCN.

OCT is a well-established, non-invasive biomedical imaging modality which can image subsurface tissue with the penetration depth of several millimeters. By obtaining and processing the coherent infrared light backscattered from the reference arm and sample arm, OCT can provide 2D cross-sectional images with high axial resolution (˜10 μm), which is 10-100 times higher than conventional medical imaging modalities (e.g., CT and MRI). Owing to the high speed of laser scanning and data processing, 3D images of the detected sample formed by numerous cross-sectional images can be obtained in real time. Because of the differences in tissue structures among the renal cortex, medulla, and calyx, OCT has the potential to distinguish different renal tissue types. Due to a 1-2 mm penetration limitation in biological tissues, studies in kidneys using OCT have mainly focused on the renal cortex. OCT can be integrated with fiber-optic catheters and endoscopes for internal imaging applications. For example, endoscopic OCT imaging has been demonstrated in the human GI tract to detect BE, dysplasia, and colon cancer. A portable, hand-held forward-imaging endoscopic OCT needle device has been developed for real-time epidural anesthesia surgery guidance. This endoscopic OCT setup holds the promise in PCN guidance.

Given the enormous accumulation of images and inter- and intra-observer variation from subjective interpretation, computer-aided automatic methods have been utilized to accurately and efficiently classify these data. In automated OCT image analysis, CNNs have been demonstrated to be promising in various applications, such as hemorrhage detection of retina versus cerebrum and tumor tissue segmentation.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic diagram of a forward-view endoscopic OCT system.

FIG. 2 is a schematic diagram of data acquisition and processing.

FIG. 3A is a diagram of an endoscope scanning a kidney sample.

FIG. 3B shows 3D OCT images, 2D cross-sectional images, and histology results of a renal cortex.

FIG. 3C shows 3D OCT images, 2D cross-sectional images, and histology results of a renal medulla.

FIG. 3D shows 3D OCT images, 2D cross-sectional images, and histology results of a renal calyx.

FIG. 4 is a table showing the average validation accuracies and their standard errors for the PT or RI model architectures after hyperparameter optimization.

FIG. 5 is a table showing the test accuracy of the best-performing model in each of the 10 testing folds.

FIG. 6A is a graph showing the ROC curve of the prediction results from kidney number 5.

FIG. 6B is a graph showing the ROC curve of the prediction results from kidney number 10.

FIG. 6C is a graph showing ROC curves for the three tissue types.

FIG. 7 is a table showing the average confusion matrix for the 10 kidneys in the 10-fold cross-testing with a score threshold of 0.333 and the average recall and precision for each type of tissue.

FIG. 8A shows class activation heatmaps for RI ResNet50 for three representative images in each tissue type

FIG. 8B shows class activation heatmaps for PT ResNet50 for three representative images in each tissue type.

FIG. 9A shows images of a blood vessel in front of a needle tip as detected by Doppler OCT.

FIG. 9B shows images of manually-labeled regions of blood vessels serving as a ground truth.

FIG. 9C shows images demonstrating the predicted vessel regions by nnU-net.

FIG. 9D shows images of superimposed OCT doppler images with manually-labeled blood vessel regions.

FIG. 9E shows images of superimposed OCT Doppler images with predicted blood vessel regions by nnU-net.

FIG. 10 is a flowchart illustrating a method showing a process for endoscopic guidance using neural networks according to one non-limiting embodiment of the present disclosure.

FIG. 11 is a schematic diagram of an apparatus according to one non-limiting embodiment of the present disclosure.

### DETAILED DESCRIPTION

Disclosed herein are embodiments for endoscopic guidance using neural networks. In an embodiment, a forward-view OCT endoscopic system images kidney tissues lying ahead of a PCN needle during PCN surgery to access the renal calyx. This may be done to remove kidney stones. In another embodiment, similar imaging is used for percutaneous renal biopsies, urine drainage, urine diversion, and other therapeutic interventions in the kidney. The embodiments provide for neural networks, for instance CNNs, which can distinguish types of renal tissue and other components. The types of renal tissue include the cortex, medulla, and calyx. Other components include blood vessels and diseased renal tissues. By distinguishing the types of renal tissue and other components, the embodiments provide for injection of a needle into the desired tissue and provide for avoidance of undesired components.

In an experiment, images of the renal cortex, medulla, and calyx were obtained from ten porcine kidneys using the OCT endoscope system. The tissue types were clearly distinguished due to the morphological and tissue differences from the OCT endoscopic images. To further improve the guidance efficacy and reduce the learning burden of the clinical doctors, a deep-learning-based, computer-aided diagnosis platform automatically classified the OCT images by the renal tissue types. A tissue type classifier was developed using the ResNet34, ResNet50, and MobileNetv2 CNN architectures. Nested cross-validation and testing were used for model selection and performance benchmarking to account for the large biological variability among kidneys through uncertainty quantification. The predictions from the CNNs were interpreted to identify the important regions in the representative OCT images used by the CNNs for the classification.

ResNet50-based CNN models achieved an average classification accuracy of 82.6%±3.0%. The classification precisions were 79%±4% for cortex, 85%±6% for medulla, and 91%±5% for calyx, and the classification recalls were 68%±11% for cortex, 91%±4% for medulla, and 89%±3% for calyx. Interpretation of the CNN predictions showed the discriminative characteristics in the OCT images of the three renal tissue types. The results validated the technical feasibility of using this novel imaging platform to automatically recognize the images of renal tissue structures ahead of the PCN needle in PCN surgery.

The following abbreviations apply:

ASIC: application-specific integrated circuit

AUC: area under the ROC curve

BD: balanced detector

BE: Barrett's esophagus

CCD: charge-coupled device

CNN: convolutional neural network

CPU: central processing unit

CT: computed tomography

DAQ: data acquisition

dB: decibel(s)

DOCT: doppler optical coherence tomography

DSP: digital signal processor

EO: electrical-to-optical

FC: fiber coupler

FOV: field of view

FPGA: field-programmable gate array

GI: gastrointestinal

GRAD-CAM: gradient-weighted class activation mapping

GRIN: gradient-index

GSM: galvanometer scanning mirror

H&E: hematoxylin and eosin

kHz: kilohertz

MEMS: microelectromechanical systems

mIoU: mean intersection-over-union

mm: millimeter(s)

MRI: magnetic resonance imaging

mW: milliwatt(s)

MZI: Mach-Zehnder interferometer

nm: nanometer(s)

OCT: optical coherence tomography

OE: optical-to-electrical

PC: polarization controller

PCN: percutaneous nephrostomy

PCNL: percutaneous nephrolithotomy

PT: pre-trained

RAM: random-access memory

ResNet: residual neural network

RF: radio frequency

RI: randomly-initialized

ROM: read-only memory

ROC: receiver operating characteristic

RX: receiver unit

SGD: stochastic gradient descent

SRAM: static RAM

SS-OCT: swept-source OCT

TCAM: ternary content-addressable memory

TX: transmitter unit

2D: two-dimensional

3D: three-dimensional

μm: micrometer(s)

°: degree(s).

Before describing various embodiments of the present disclosure in more detail by way of exemplary description, examples, and results, it is to be understood that the present disclosure is not limited in application to the details of methods and compositions as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-70, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. A reference to degrees such as 1 to 90 is intended to explicitly include all degrees in the range. A reference to the number of unit cells in a sub-array panel, such as 4-256, 4-400, or 4-676, is intended to include all whole numbers (positive integers) within each range.

In certain embodiments, the element spacing of the disclosed SWGA array units can be in a range of about 0.6λₒ to about 0.5λₒ (in the azimuth plane), providing a reduction of from about 50% to about 58% vs. a conventional spacing of 1.2λₒ, thereby enabling a 1D e-scanning range in a range of from about 84° (±42°) up to at about 180° (±90°) in the azimuth plane perpendicular to the waveguide axis. For example, the element spacing may be from 0.6λₒ, to about 0.59λₒ, to about 0.58λₒ, to about 0.57λₒ, to about 0.56λₒ, to about 0.55λₒ, to about 0.54λₒ, to about 0.53λₒ, to about 0.52λₒ, to about 0.51λₒ, to about 0.50λₒ, or fractional portions thereof, thereby enabling a 1D e-scanning range of from about 84° (±42°), to about 86° (±43°), to about 88° (±44°), to about 90° (±45°), to about 92° (±46°), to about 94° (±47°), to about 96° (±48°), to about 98° (±49°), to about 100° (±50°), to about 102° (±51°), to about 104° (±52°), to about 106° (±53°), to about 108° (±54°), to about 110° (±55°), to about 112° (±56°), to about 114° (±57°), to about 116° (±58°), to about 118° (±59°), to about 120° (±60°), to about 122° (±61°), to about 124° (±62°), to about 126° (±63°), to about 128° (±64°), to about 130° (±65°), to about 132° (±66°), to about 134° (±67°), to about 136° (±=68°), to about 138° (±69°), to about 140° (±70°), to about 142° (±71°), to about 144° (±72°), to about 146° (±73°), to about 148° (±74°), to about 150° (±75°), to about 152° (±76°), to about 154° (±77°), to about 156° (±78°), to about 158° (±79°), to about 160° (±80°), to about 162° (±81°), to about 164° (±82°), to about 166° (±83°), to about 168° (±84°), to about 170° (±85°), to about 172° (±86°), to about 174° (±87°), to about 176° (±88°), to about 178° (±89°), to at about 180° (±90°). Cross-polarization isolation may be within a range of about −55 dB to about −70 dB, but will generally be within a range of about −60 dB to about −70 dB.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error. Further, in this detailed description, each numerical value (e.g., degrees or frequency) should be read once as modified by the term “about” (unless already expressly so modified), and then read again as not so modified unless otherwise indicated in context. As noted, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. The use of the term “about” may mean a range including ±10% of the subsequent number unless otherwise stated.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement (e.g., degrees, frequency, width, length, etc.).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

### System

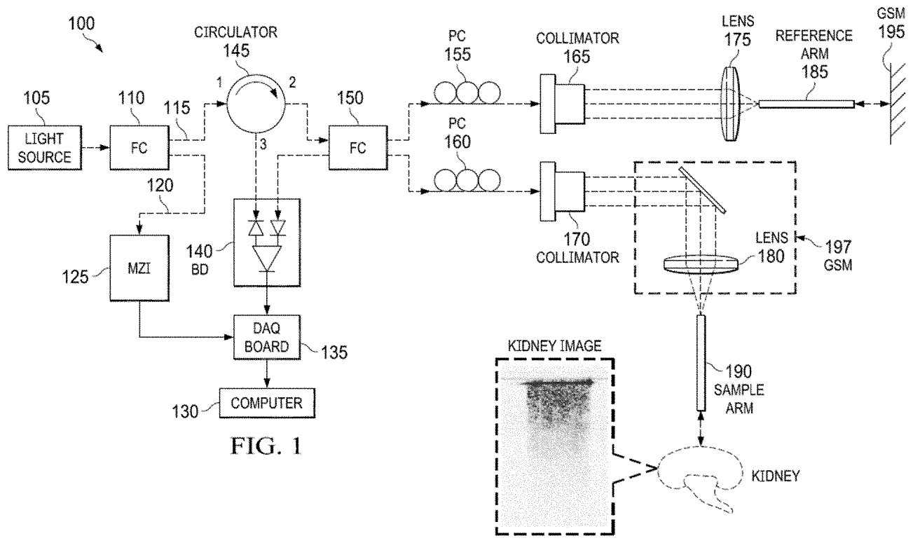

FIG. 1 is a schematic diagram of a forward-view endoscopic OCT system 100. The forward-view endoscopic OCT system 100 is based on an SS-OCT system and comprises a light source 105, an FC 110, a top path 115, a bottom path 120, an MZI 125, a computer 130, a DAQ board 135, a BD 140, a circulator 145, an FC 150, a PC 155, a PC 160, a collimator 165, a collimator 170, a lens 175, a lens 180, a reference arm 185, a sample arm 190, a GSM 195, and a GSM 197. The lenses 175, 180 are GRIN rod lenses with a 1.3 mm diameter. The reference arm 185 and the sample arm 190 form a Michelson interferometer. The forward-view endoscopic OCT system 100 could have an inner diameter of 0.1-8.0 mm, a length of 5-400 mm, and a view angle of 0-50°. With a protective steel tubing, the forward-view endoscopic OCT system 100 has an outer diameter of about 0.35-10.00 mm.

The light source 105 generates a laser beam with a center wavelength of 1300 nm and a bandwidth of 100 nm. The wavelength-swept frequency (A-scan) rate is 200 kHz with an ˜25 mW output power. The FC 110 splits the laser beam into a first beam with 97% of the whole laser power on the top path 115 and a second beam with 3% of the whole laser power on the bottom path 120. The second beam delivers into the MZI 125 for the MZI 125 to generate a frequency clock signal. The frequency clock signal triggers an OCT sampling procedure and passes to the DAQ board 135. The first beam passes to the circulator 145, which runs only in one direction. Therefore, the light entering port 1 only emits from port 2, and then it evenly splits towards the reference arm 185 and the sample arm 190. Backscattered light from both the reference arm 185 and the sample arm 190 form interference fringes at the FC 150 and transmit to the BD 140. The interference fringes from different depths received by the BD 140 are encoded with different frequencies. The BD 140 transmits an output signal to the DAQ board 135 and the computer 130 for processing. Cross-sectional information can be obtained through a Fourier transform of the interference fringes.

In the experiment, the lenses 175, 180 were stabilized in front of GSMs 195, 197. The proximal GRIN lens entrance of the endoscope was placed close to the focal plane of the objective lens. The GRIN lens can preserve the spatial relationship between the entrance and the output (distal end) and further to the sample. Therefore, one or two directional scanning can be readily performed on the proximal GRIN lens surface to create 2D or 3D images. In addition, the same GRIN rod lens was put in the light path of the reference arm 185 for the purpose of compensating light dispersion and expanding the length of the reference arm 185. The PCs 155, 160 decreased background noise. The forward-view endoscopic OCT system 100 had an axial resolution of ˜11 μm and lateral resolution of ˜20 μm in tissue. The lateral imaging FOV was around 1.25 mm. The sensitivity of the forward-view endoscopic OCT system 100 was optimized to 92 dB and calculated using a silver mirror with a calibrated attenuator.

### Data Acquisition

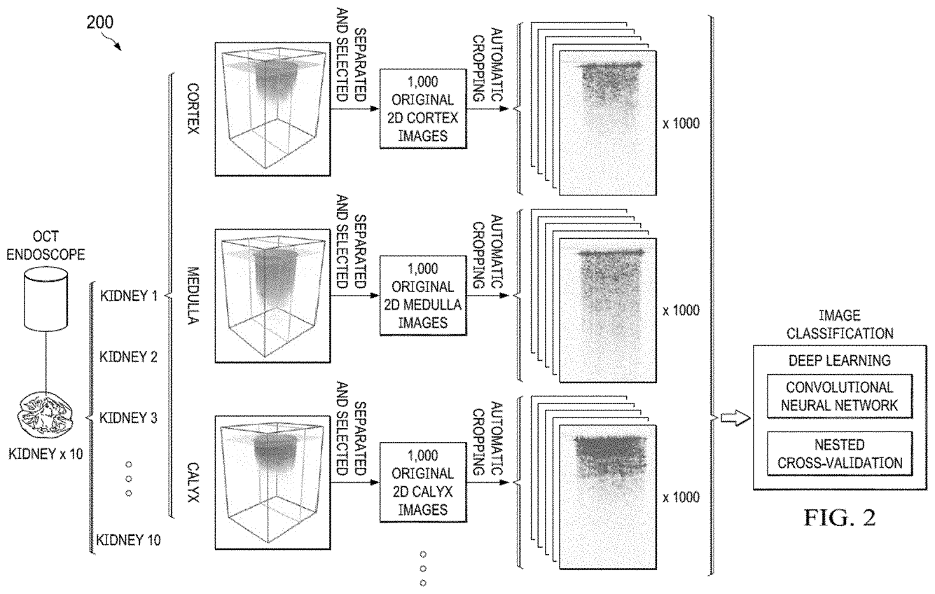

Ten fresh porcine kidneys were obtained from a local slaughterhouse. The cortex, medulla, and calyx of the porcine kidneys were exposed and imaged in the experiment. Renal tissue types can be identified from the anatomic appearance. The forward-view endoscopic OCT system 100 was placed against different renal tissues for image acquisition. To mimic a clinical situation, some force was applied while imaging the ex-vivo kidney tissues to generate tissue compression. 3D images of 320×320×480 pixels on X, Y and Z axes (Z presents the depth direction) were obtained with the pixel size of 6.25 μm on all three axes. Therefore, the size of the original 3D images is 2.00 mm×2.00 mm×3.00 mm. For every kidney sample, at least 30 original 3D OCT images were obtained for each tissue type, and each 3D tissue scanning took no more than 2 seconds. Afterwards, the original 3D images were separated to 2D cross-sectional images as shown in FIG. 2. FIG. 2 is a schematic diagram 200 of data acquisition and processing.

Since the GRIN lens is cylindrical, the 3D OCT images obtained were also in the cylindrical shape. Therefore, not all of the 2D cross-sectional images contained the same structural signal of the kidney. Only the 2D images with sufficient tissue structural information (cross-sectional images close to the center of the 3D cylindrical structures) were subsequently selected and utilized for the image preprocessing. At the end of imaging, tissues of cortex, medulla, and calyx of the porcine kidneys were excised and processed for histology to compare with corresponding OCT results. The tissues were fixed with 10% formalin, embedded in paraffin, sectioned (4 μm thick) and stained with H&E for histological analysis. Images were taken by Keyence Microscope BZ-X800.

Although the three tissue types showed different imaging features for visual recognition, it will take time and expertise for doctors to differentiate them during surgeries. In order to improve the efficiency, we developed deep learning methods for automatic tissue classification based on the imaging data. In total, ten porcine kidneys were imaged in this study. For each kidney, 1,000 2D cross-sectional images were obtained for each cortex, medulla, and calyx. For the purpose of convenient analysis and increasing the speed of deep-learning process of the OCT images, a custom MATLAB algorithm was designed to recognize the surface of the kidney tissue on the 2D cross-sectional images. The algorithm automatically cropped the images from the size of 320×480 to 235×301. Therefore, all the 2D cross-sectional images have the same dimensions and cover the same FOV before deep-learning processing.

### CNN Training

A CNN was used to classify the images of the renal cortex, medulla, and calyx. ResNet34, ResNet50, and MobileNetv2 were tested using Tensorflow 2.3 in open-ce version 0.1.

Pre-trained ResNet50 and MobileNetv2 models on the ImageNet dataset were imported. The output layer of the models was changed to one containing 3 softmax output neurons for cortex, medulla, and calyx. The input images were preprocessed by resizing to the 224×224 resolution, replicating the input channel to 3 channels, and scaling the pixel intensities to [−1, 1]. Model fine-tuning was conducted in two stages. First, the output layer was trained with all the other layers frozen. The optimizer, SGD, was used with a learning rate of 0.2, a momentum of 0.3, and a decay of 0.01. Then, the entire model was unfrozen and trained. The SGD with Nesterov momentum optimizer was used with a learning rate of 0.01, a momentum of 0.9, and a decay of 0.001. Early stopping with a patience of 10 and a maximum number of epochs 50 was used for the Pre-trained ResNet50. Early stopping with a patience of 20 and a maximum number of epochs 100 was used for MobileNetv2.

The ResNet34 and ResNet50 architectures were also trained using randomly initialized weights. ResNet34 was obtained. The mean pixel in the training dataset was used to center the training, validation, and test datasets. The input layer was modified to accept only one input channel in the OCT images and the output layer was changed for the classification of the three tissue types. For ResNet50, the optimizer SGD with Nesterov momentum with learning rate 0.01, momentum 0.9, and decay 0.01 was used. ResNet50 was trained with a maximum of 50 epochs, early stopping with a patience of 10, and a batch size of 32. For ResNet34, the Adam optimizer was used with learning rate 0.001, beta1 0.9, beta2 0.9999 and epsilon 1E-7. ResNet34 was trained with a maximum of 200 epochs, early stopping with a patience of 10, and a batch size of 512.

### Validation and Testing

A nested cross-validation and testing procedure was used to estimate the validation performance and the test performance of the models across the 10 kidneys with uncertainty quantification. The pseudo-code of the nested cross-validation and testing is shown below.

```
# 10-fold cross-testing loop
for kidney i in the 10 kidneys do
  Hold out kidney i in the test set
  # model optimization loop
  for each model configuration do
    # 9-fold cross-validation loop
  for kidney j in the remaining 9 kidneys do
    Use kidney j as the validation set
    Train a model using the remaining 8 kidneys as the training set
    Benchmark the validation performance using kidney j
    end for
    Estimate the mean validation accuracy and its standard error
  end for
  Select the best model configuration based on the validation performance
  Train a model with the selected configuration using the 9 kidneys
  Benchmark the test performance of this model using kidney i
end for
Summarize the test performance of this procedure
```

In the 10-fold cross-testing, one kidney was selected in turn as the test set. In the 9-fold cross-validation, the remaining nine kidneys were partitioned 8:1 between the training set and the validation set. Each kidney had a total of 3,000 images, including 1,000 images for each tissue type. The validation performance of a model was tracked based on its classification accuracy on the validation kidney. The classification accuracy is the percentage of correctly labeled images out of all 3,000 images of a kidney.

The 9-fold cross-validation loop was used to compare the performance of ResNet34, ResNet50, and MobileNetv2, and optimize the key hyperparameters of these models, such as pre-trained versus randomly initialized weights, learning rates, and number of epochs. The model configuration with the highest average validation accuracy was selected for the cross-testing loop. The cross-testing loop enabled iterative benchmarking of the selected model across all 10 kidneys, giving a better estimation of generalization error with uncertainty quantification.

GRAD-CAM was used to explain the predictions of a selected CNN model by highlighting the important regions in the image for the prediction outcome.

### OCT Imaging of Different Renal Tissues

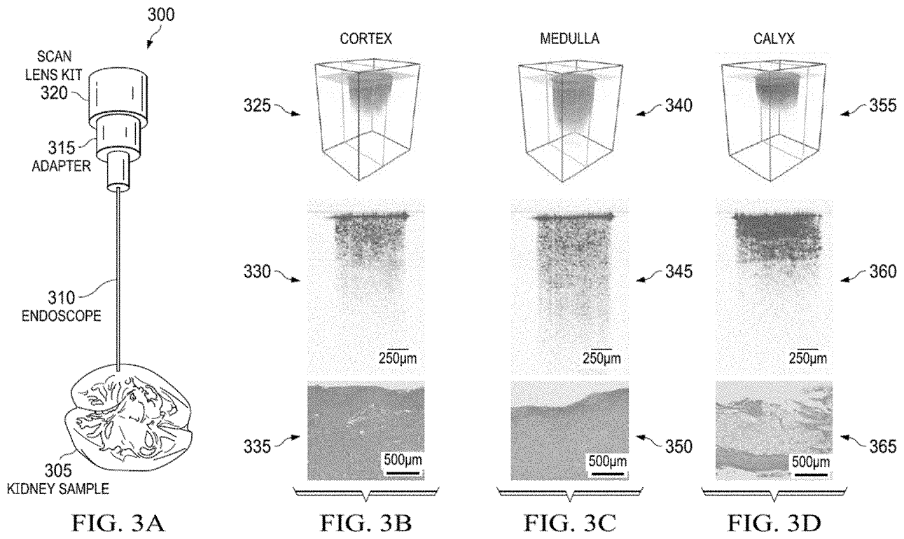

FIG. 3A is a diagram 300 of an endoscope 310 scanning a kidney sample 305. An adapter 315 stabilizes the endoscope 310 in front of an OCT scan lens kit 320. The kidney sample 305 shows different tissue types. Renal cortex is the brown tissue on the edge of the kidney sample 305. Medulla can be recognized by its red renal pyramid structures distributed on the inner side of the cortex. Calyx is featured by its obvious white structure in the central portion of the kidney sample 305. Three tissue types were imaged respectively following the procedure described above.

FIG. 3B shows 3D OCT images 325, 2D cross-sectional images 330, and histology results 335 of a renal cortex. FIG. 3C shows 3D OCT images 340, 2D cross-sectional images 345, and histology results 350 of a renal medulla. FIG. 3D shows 3D OCT images 355, 2D cross-sectional images 360, and histology results 365 of a renal calyx. FIGS. 3B-3D are featured with different imaging depth and brightness.

The renal calyx in FIG. 3B has the shallowest imaging depth, but the tissue close to the surface shows the highest brightness and density. The renal cortex in FIG. 3C and the renal medulla in FIG. 3D both present relatively homogeneous tissue structures in the 3D OCT images 340 and 355, and the imaging depth of the renal medulla in FIG. 3C is larger than the renal cortex in FIG. 3D. Furthermore, compared to the renal cortex in FIG. 3B and the renal medulla in FIG. 3D, the renal calyx in FIG. 3D was featured with horizontal stripes and a layered structure. The transitional epithelium and fibrous tissue in the renal calyx in FIG. 3D may explain the strip-like structures and significantly higher brightness in comparison to the other two renal tissues. This is the significant part for PCN insertion because the goal of PCN is to reach the calyx precisely. These imaging results demonstrated the feasibility of distinguishing the renal cortex, medulla, and calyx with the endoscopic OCT system.

### CNN Development and Benchmarking Results

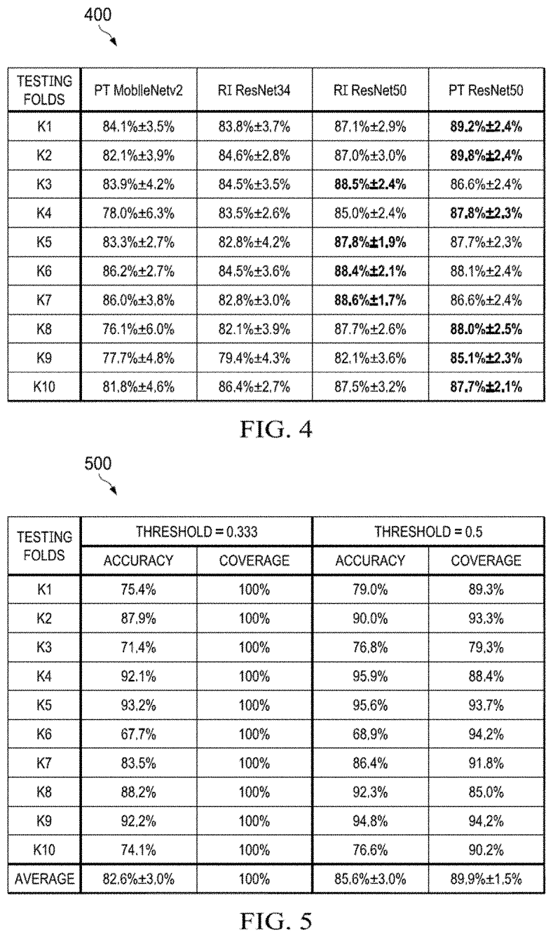

FIG. 4 is a table 400 showing the average validation accuracies and their standard errors for the PT or RI model architectures after hyperparameter optimization. RI MobileNetv2 frequently failed to learn, so only the PT MobileNetv2 model was used. The PT ResNet50 models outperformed the RI ResNet50 models in 6 of the 10 testing folds, which indicated only a small boost by the pre-training on ImageNet. For all the 10 testing folds, the validation accuracies of the ResNet50 models were significantly higher than those of the MobileNetv2 and ResNet34 models. Thus, the characteristic patterns of the three kidney tissues may require a deep CNN architecture to be recognized.

FIG. 5 is a table 500 showing the test accuracy of the best-performing model in each of the 10 testing folds. The output layer of the CNN models estimated three softmax scores that summed up to 1.0 for the three tissue types. When the category with the highest softmax score was selected for an image (i.e., a softmax score threshold of 0.333 to make a prediction), the CNN model made a prediction for every image (100% coverage) at a mean test accuracy of 82.6%. This was substantially lower than the mean validation accuracy of 87.3%, which suggested the overfitting to the validation set by the hyperparameter tuning and early stopping. The classification accuracy can be increased at the expense of lower coverage by increasing the softmax score threshold, which allowed the CNN model to make only confident classifications. When the softmax score threshold was raised to 0.5, 89.9% of the images on average were classified to a tissue type and the mean classification accuracy increased to 85.6%±3.0%. For the uncovered images, the doctors can make a prediction with the help of other imaging modality and their clinical experience.

There was substantial variability in the test accuracy among different kidneys. While three kidneys had test accuracies higher than 92% (softmax score threshold of 0.333), the kidney in the sixth fold had the lowest test accuracy of 67.7%. Therefore, the current challenge in the image classification mainly comes from the anatomic differences among the samples.

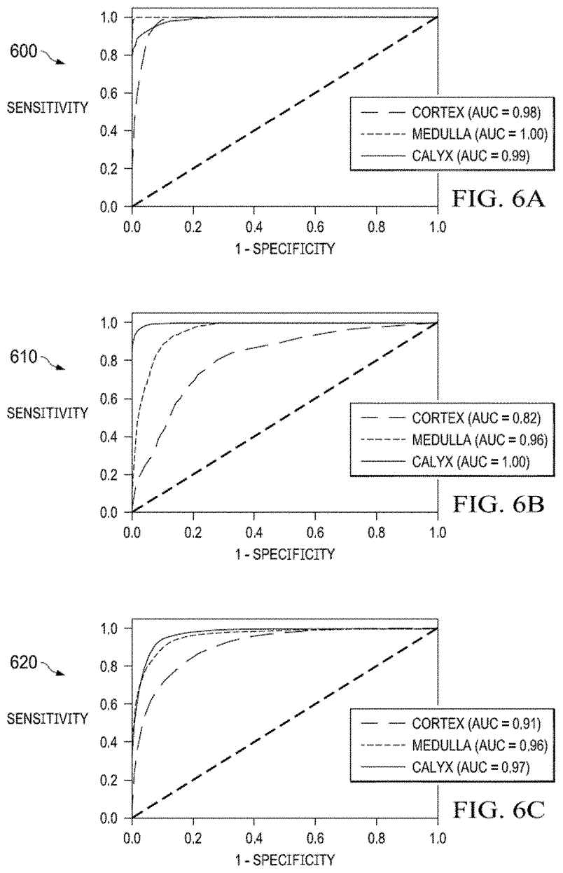

FIG. 6A is a graph 600 showing the ROC curve of the prediction results from kidney number 5. FIG. 6B is a graph 610 showing the ROC curve of the prediction results from kidney number 10. It is clear that the prediction of kidney number 5 is much more accurate than that of kidney number 10. The nested cross-validation and testing procedure was designed to simulate the real clinical setting in which the CNN models trained on one set of kidneys need to perform well on a new kidney unseen by the CNN models until the operation. When a CNN model was trained on a subset of images from all kidneys and validated on a separate subset of images from all kidneys in cross-validation as opposed to partitioning by kidneys, it achieved accuracies over 99%. This suggested that the challenge of image classification mainly stemmed from the biological differences between different kidneys. The generalization of the CNN models across kidneys can be improved by expanding the dataset with kidneys at different ages or physical conditions to represent different structural and morphological features.

FIG. 6C is a graph 620 showing average ROC curves for the three tissue types. The AUC was 0.91 for the cortex, 0.96 for the medulla, and 0.97 for the calyx.

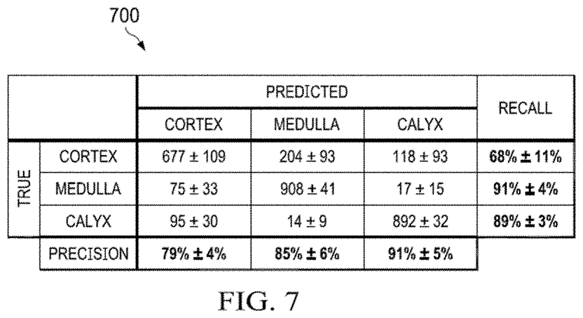

FIG. 7 is a table 700 showing the average confusion matrix for the 10 kidneys in the 10-fold cross-testing with a score threshold of 0.333 and the average recall and precision for each type of tissue. The cortex was the most challenging tissue type to be classified correctly and was sometimes mixed up with the medulla. From the original images, it was found that the penetration depths of the medulla were much larger than the cortex in seven of the ten imaged kidneys. These differences were insignificant in three other samples. This may explain the challenging classification between the cortex and the medulla.

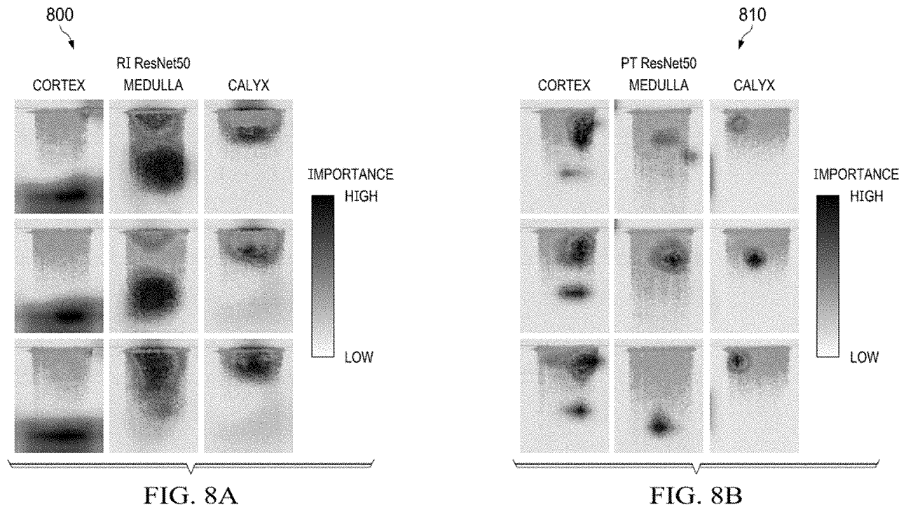

FIG. 8A shows class activation heatmaps 800 for RI ResNet50 for three representative images in each tissue type. FIG. 8B shows class activation heatmaps 810 for PT ResNet50 for three representative images in each tissue type. The class activation heatmaps 800 and 810 help demonstrate how a CNN model classified different renal types. The models and the representative images were selected from the fifth testing fold. The RI ResNet50 model performed the classification by focusing on the lower part of the images of the cortex, to both the lower part and near the upper part of the medulla images, and to the area of the calyx images with high intensity near the needle tip. The PT ResNet50 model focused on both the upper part and the lower part of the cortex images, on the middle part or the lower part of the medulla images, and on the region close to the needle tip of the calyx images. Compared to the RI ResNet50 model, the PT ResNet50 model focused more on the signal regions. The class activation heatmaps 800 and 810 provided an intuitive explanation of the classification basis for the two CNN models.

### Detecting Blood Vessels

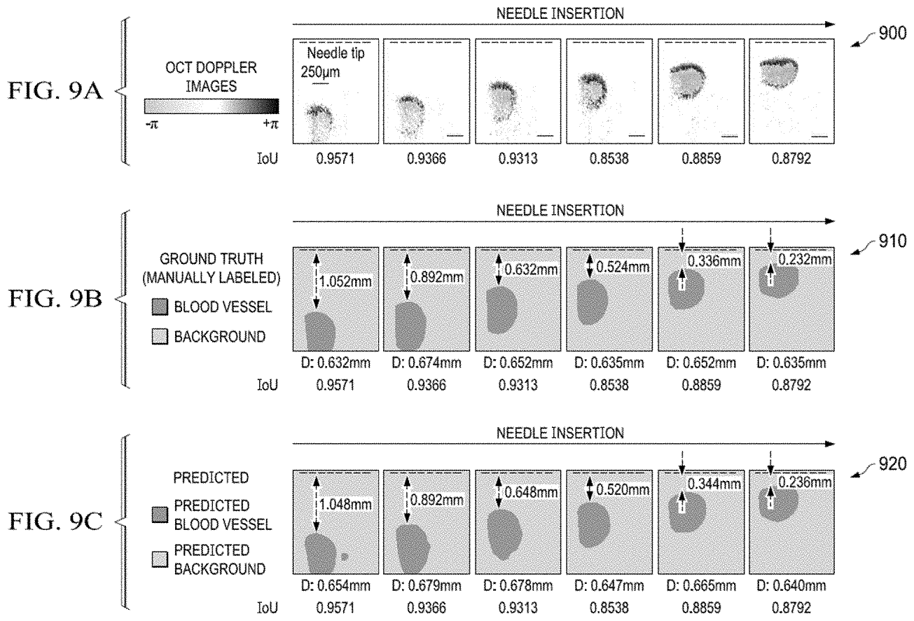

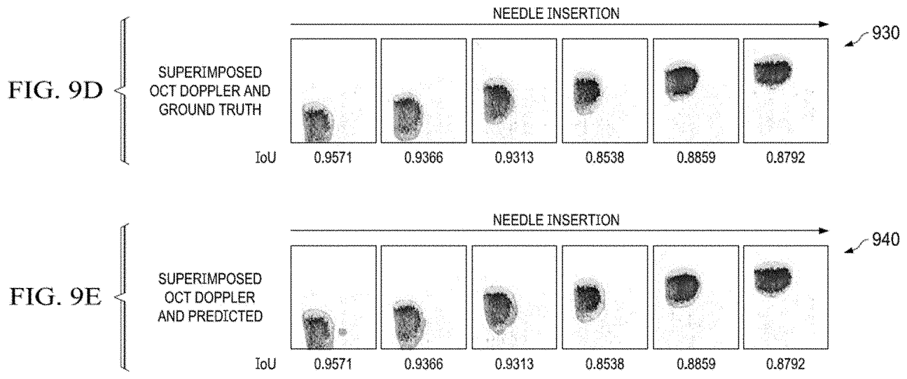

FIGS. 9A-9E show segmentation of blood vessels from Doppler OCT images. FIG. 9A shows images 900 of a blood vessel in front of a needle tip as detected by Doppler OCT. FIG. 9B shows images 910 of manually-labeled regions of blood vessels serving as a ground truth. The dashed line indicates the location of the needle tip, and “D” is the diameter of the blood vessel. FIG. 9C shows images 920 demonstrating the predicted vessel regions by nnU-net. FIG. 9D shows images 930 of superimposed OCT doppler images with manually-labeled blood vessel regions. FIG. 9E shows images 940 of superimposed OCT Doppler images with predicted blood vessel regions by nnU-net.

The real-time blood vessel detection of the forward imaging OCT/DOCT needle in another 5 perfused human kidneys was demonstrated. During the insertion of the OCT needle into the kidney in the PCN procedure, the blood vessels in front of the needle tip were detected by Doppler OCT. FIG. 9A demonstrates a surgical scenario in which the probe encountered and compressed a ˜0.65 mm-diameter (lumen) blood vessel. The vessel was first detected at ˜1 mm distance, the upper boundary of the vessel to the needle tip, in front of the probe. The vessel was tracked as the needle advanced, and the probe was stopped prior to penetrating the vessel. Further advancing the needle compressed the vessel as shown in the far-right image in the images 900. The results demonstrated the feasibility of using DOCT imaging to detect the at-risk blood vessels during the PCN procedure using the hand-held OCT probe, without any impact of motion artifacts.

To improve the accuracy of image segmentation, a novel nnU-net framework was trained and tested using 100 2D Doppler OCT images. The blood vessels in these 100 images were first manually labeled to mark the blood vessel regions as shown in FIGS. 9B and 9D. The training and validation sets with 60 images were used to develop the nnU-net model. The segmentation accuracy of the nnU-net model was benchmarked using 40 test images. The mIoU of the predicted blood vessel pixels reached 88.46% as shown in FIGS. 9C and 9E. These new preliminary results demonstrate that with the deep-learning model, a greater than 88% mIoU can be achieved for 2D Doppler OCT images.

After obtaining the predicted regions by nnU-net as shown in FIG. 9C, an automatic image processing algorithm was developed to measure the size of the blood vessel and the distance from the needle tip. The region/area of the blood vessel was quantified by counting the pixels in the predicted regions, and the diameter was fitted by using the circular area formula because the irregular shape of the blood vessel is due to tissue compression. The distance was measured from the preset needle tip to the predicted most upper surface of the blood vessel. Compared to the ground truth shown in FIG. 9B, an accuracy (1−|Predicted−Ground Truth|/Ground Truth) of 98.7%±0.67% was achieved for distance prediction and an accuracy of 97.85%±1.34% for blood vessel diameter estimation by this algorithm.

These preliminary data clearly demonstrated at least three favorable outcomes. First, the thin-diameter forward imaging OCT/DOCT needle can detect the blood vessels in front of the needle tip in real time in the human kidney. Second, the newly developed nnU-net model can achieve >88% mIoU for 2D Doppler OCT images. Third, the size and location of blood vessel can be accurately predicted. Thus, this showed a viable approach to preventing accidental blood vessel ruptures.

### CONCLUSION

The feasibility of an OCT endoscopic system for PCN surgery guidance was investigated. Three porcine kidney tissue types, the cortex, medulla and calyx, were imaged. These three kidney tissues show different structural features, which can be further used for tissue type recognition. To increase the image recognition efficiency and reduce the learning burden of the clinical doctors, CNN methods were developed and evaluated for image classification and recognition. ResNet50 had the best performance compared to ResNet34 and PT MobileNetv2 and achieved an average classification accuracy of 82.6%±3.0%.

The porcine kidneys samples were obtained from a local slaughterhouse without controlling the sample preservation and time after death. Biological changes may have occurred in the ex-vivo kidneys, including collapse of some structures of nephrons such as the renal tubules. This may have made the tissue recognition more difficult, especially the classification between the cortex and the medulla. Characteristic renal structures in the cortex can be clearly imaged by OCT in both well-preserved ex-vivo human kidneys and living kidneys and verified in an ongoing study in a lab using well-preserved human kidneys. Additionally, nephron structures distributed in the renal cortex and the medulla are different. These additional features in the renal cortex and the medulla will improve the recognition of these two tissue types and increase the classification accuracy of future CNN models when imaging in-vivo samples or well-preserved ex-vivo samples. The study established the feasibility of automatic tissue recognition using CNN and provided information for the model selection and hyper-parameter optimization in future CNN model development using in-vivo pig kidneys and well-preserved ex-vivo human kidneys.

For translating the proposed OCT probe into clinics, the endoscope will be assembled with appropriate diameter and length into the clinically-used PCN needle. In current PCN punctures, a trocar needle is inserted into the kidney. Since the trocar has a hollow structure, the endoscope can be fixed within the trocar needle. Then the OCT endoscope can be inserted into the kidney together with the trocar needle. After the trocar needle tip arrives at the destination (such as the kidney pelvis), we will withdraw the OCT endoscope from the trocar needle and other surgical processes can be continued. During the whole puncture, no extra invasiveness will be caused. Since the needle will keep moving during the puncture, there will be a tight contact between the needle tip and the tissue. Therefore, the blood, if any, will not accumulate in front of the needle tip. From previous experience in the in-vivo pig experiment guiding the epidural anesthesia using the OCT endoscope, the presence of blood is not a substantial issue. The diameter of the GRIN rod lens used in the study was 1.3 mm. In the future, the current setup will be improved with a smaller GRIN rod lens that can be fit inside the 18-gauge PCN needle, which is clinically used in the PCN puncture. Furthermore, the GSM device will be miniaturized based on MEMS technology, which will enable ease of operation and is important for translating the OCT endoscope to clinical applications. The current employed OCT system has a scanning speed up to 200 kHz, and the 2D tissue images in front of the PCN needle can be provided to surgeons in real time. Using ultra-high-speed laser scanning and a data processing system, 3D images of the detected sample can be obtained in real time. In the next step, 3D images that further improve classification accuracy may be acquired because of the added information content in 3D images.

### Exemplary Method

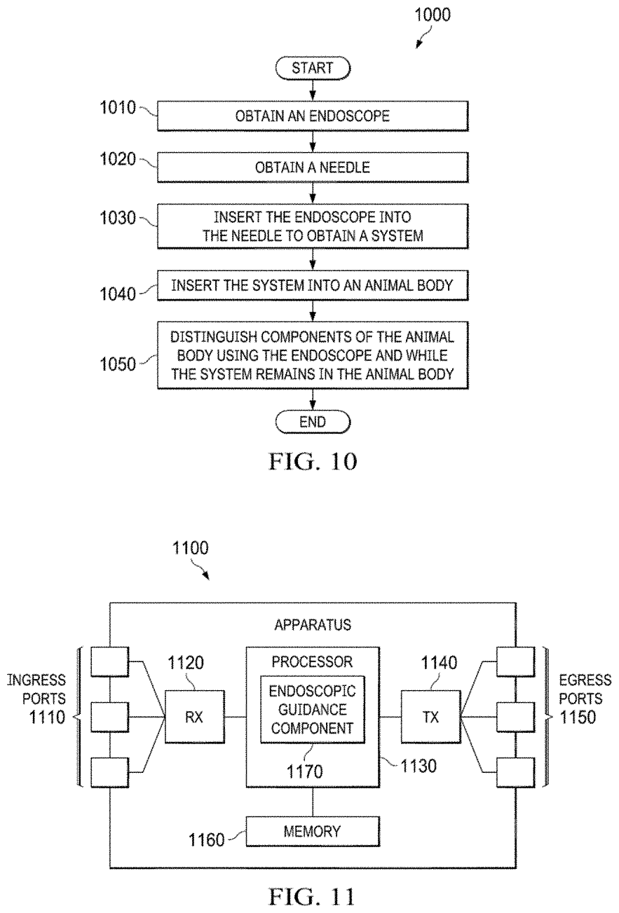

FIG. 10 is a flowchart illustrating a method 1000 showing a process for endoscopic guidance using neural networks according to one non-limiting embodiment of the present disclosure. At step 1010, an endoscope is obtained. At step 1020, a needle is obtained. At step 1030, the endoscope is inserted into the needle to obtain a system. At step 1040, the system is inserted into an animal body. Finally, at step 1050, components of the animal body are distinguished using the endoscope and while the system remains in the animal body.

The method 1000 may comprise additional embodiments. For instance, the method 1000 further comprises training a CNN to distinguish the components. The method 1000 further comprises further training the CNN to distinguish among a cortex of a kidney, a medulla of the kidney, and a calyx of the kidney. The method 1000 further comprises further training the CNN to distinguish blood vessels from other components. The method 1000 further comprises incorporating the CNN into the endoscope. The CNN comprises an input layer, a convolutional layer, a max-pooling layer, a flatten layer, dense layers, and an output layer. The animal body is a human body. The method 1000 further comprises further distinguishing a calyx of a kidney from a cortex of the kidney and a medulla of the kidney; inserting, based on the distinguishing, the system into the calyx; and removing the endoscope from the system to obtain the needle. The method 1000 further comprises further distinguishing the calyx from a blood vessel; and avoiding contact between the system and the blood vessel. The method 1000 further comprises removing kidney stones while the needle remains in the calyx. The method 1000 further comprises inserting, based on the distinguishing, the system into a kidney of the animal body; and obtaining a biopsy of the kidney. The system is a forward-view endoscopic OCT system.

### Exemplary Computing Apparatus

FIG. 11 is a schematic diagram of an apparatus 1100 according to one non-limiting embodiment of the present disclosure. The apparatus 1100 may implement the disclosed embodiments. The apparatus 1100 comprises ingress ports 1110 and an RX 1120 to receive data; a processor 1130, or logic unit, baseband unit, or CPU, to process the data; a TX 1140 and egress ports 1150 to transmit the data; and a memory 1160 to store the data. The apparatus 1100 may also comprise OE components, EO components, or RF components coupled to the ingress ports 1110, the RX 1120, the TX 1140, and the egress ports 1150 to provide ingress or egress of optical signals, electrical signals, or RF signals.

The processor 1130 is any combination of hardware, middleware, firmware, or software. The processor 1130 comprises any combination of one or more CPU chips, cores, FPGAs, ASICs, or DSPs. The processor 1130 communicates with the ingress ports 1110, the RX 1120, the TX 1140, the egress ports 1150, and the memory 1160. The processor 1130 comprises an endoscopic guidance component 1170, which implements the disclosed embodiments. The inclusion of the endoscopic guidance component 1170 therefore provides a substantial improvement to the functionality of the apparatus 1100 and effects a transformation of the apparatus 1100 to a different state. Alternatively, the memory 1160 stores the endoscopic guidance component 1170 as instructions, and the processor 1130 executes those instructions.

The memory 1160 comprises any combination of disks, tape drives, or solid-state drives. The apparatus 1100 may use the memory 1160 as an over-flow data storage device to store programs when the apparatus 1100 selects those programs for execution and to store instructions and data that the apparatus 1100 reads during execution of those programs. The memory 1160 may be volatile or non-volatile and may be any combination of ROM, RAM, TCAM, or SRAM.

A computer program product may comprise computer-executable instructions for storage on a non-transitory medium and that, when executed by a processor, cause an apparatus to perform any of the embodiments. The non-transitory medium may be the memory 1160, the processor may be the processor 1130, and the apparatus may be the apparatus 1100.

While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method comprising:
obtaining an endoscope;
obtaining a needle;
inserting the endoscope into the needle to obtain a system;
inserting the system into an animal body; and
distinguishing components of the animal body using the endoscope and while the system remains in the animal body.

2. The method of claim 1, further comprising training a convolutional neural network (CNN) to distinguish the components.

3. The method of claim 2, further comprising further training the CNN to distinguish among a cortex of a kidney, a medulla of the kidney, and a calyx of the kidney.

4. The method of claim 2, further comprising further training the CNN to distinguish blood vessels from other components.

5. The method of claim 2, further comprising incorporating the CNN into the endoscope.

6. The method of claim 2, wherein the CNN comprises an input layer, a convolutional layer, a max-pooling layer, a flatten layer, dense layers, and an output layer.

7. The method of claim 1, wherein the animal body is a human body.

8. The method of claim 1, further comprising:
further distinguishing a calyx of a kidney from a cortex of the kidney and a medulla of the kidney;
inserting, based on the distinguishing, the system into the calyx; and
removing the endoscope from the system to obtain the needle.

9. The method of claim 8, further comprising:
further distinguishing the calyx from a blood vessel; and
avoiding contact between the system and the blood vessel.

10. The method of claim 8, further comprising removing kidney stones while the needle remains in the calyx.

11. The method of claim 1, further comprising:
inserting, based on the distinguishing, the system into a kidney of the animal body; and
obtaining a biopsy of the kidney.

12. The method of claim 1, wherein the system is a forward-view endoscopic optical coherence tomography (OCT) system.

13. A system comprising:
a needle; and
an endoscope inserted into the needle and configured to:
store a convolutional neural network (CNN);
distinguish among a cortex of a kidney of an animal body, a medulla of the kidney, and a calyx of the kidney using the CNN; and
distinguish between vascular tissue and non-vascular tissue in the animal body using the CNN.

14. The system of claim 13, wherein the CNN comprises an input layer, a convolutional layer, a max-pooling layer, a flatten layer, dense layers, and an output layer.

15. The system of claim 13, wherein the animal body is a human body.

16. The system of claim 13, wherein the system is a forward-view endoscopic optical coherence tomography (OCT) system.

17. The system of claim 13, wherein the endoscope has a diameter of about 1.3 millimeters (mm).

18. The system of claim 13, wherein the endoscope has a length of about 138.0 millimeters (mm).

19. The system of claim 13, wherein the endoscope is configured to have a view angle of 11.0°.

20. The system of claim 13, wherein the needle is configured to remove a kidney stone from the kidney or obtain a biopsy of the kidney.
